(19) United States
(12) Patent Application Publication
(10) Pub. No.: US 2024/0345021 A1
(43) Pub. Date: Oct. 17, 2024

(54) Handheld Device for Early Detection of Disease, Biological, Chemical, and Pathogenic Agents

(71) Applicant: Richard POSTREL, Miami Beach, FL (US)

(72) Inventor: Richard POSTREL, Miami Beach, FL (US)

(21) Appl. No.: 18/542,582

(22) Filed: Dec. 15, 2023

Related U.S. Application Data
(63) Continuation of application No. PCT/US22/33697, filed on Jun. 15, 2022.
(60) Provisional application No. 63/210,938, filed on Jun. 15, 2021.

(51) Int. Cl. G01N 27/414; G01N 33/543

(52) U.S. Cl. CPC G01N 27/4141; G01N 33/5438

(57) ABSTRACT

The present invention provides a portable handheld medical screening device designed to facilitate health, medical research, and health-security screening measures at hospitals, emergency rooms, medical facilities, offices, meeting rooms, sports training facilities, health clubs, restaurants, small shops and for service providers, etc., i.e., places associated with healthcare and interactions between persons unknown to each other in settings where disease, public health, research data collection, disease contagion, and/or disease detection and/or monitoring are a concern. The invention provides several formats of self-contained devices with flexible attachment that collects ambient emissions, especially volatile compounds, produced by a subject (medical patient or one or more persons interacting with each other). In a clinical setting, the device monitors gas compounds produced by the subject, analyzes the monitored gases to determine patterns of emitted gases, compares these patterns to libraries of patterns associated with a disease or condition, and reports the associations.

The present invention is designed to facilitate health, medical research, and security screening measures at hospitals, emergency rooms, medical facilities, offices, meeting rooms, sports training facilities, restaurants, small shops and service providers, etc., I.e., places associated with healthcare and interactions between persons unknown to each other in settings where disease, public health, research data collection, disease contagion, and disease detection are a concern. A handheld self-contained device uses a flexible probe attachment to sample ambient air in a closed environment and/or the volatile compounds produced by one or more persons in a zone or area where contagion may be a concern. Used in a clinical setting the device monitors gas compounds produced by a patient or subject of interest, analyzes the monitored gases for emitted gas patterns, compares these patterns to one or more libraries of patterns associated with disease and reports any disease associations. The operation of the device can be set to screen for a limited class of diseases or for screening against the consolidated library of all associated diseases. The assay device is designed to be light weight and manageable by a single operator. The operator requires no intricate training; minimal requirements involve merely using a hand to position the end of the probe to the zone of interest around the subject, depressing the trigger while maintaining probe position, waiting for a signal to stop, and withdrawing the probe to a neutral position. No chemical or biologic reagents are necessary greatly simplifying operation and amount of training.

In a clinical setting, e.g., in a hematology/oncology specialist office, the device may compare the patient's results to a library of oncologic diseases and libraries of diseases that complicate oncologic treatments. In an emergency room or in a general practitioner setting, the screen may be set for comparison with a full range of diseases and conditions, including contagious disease, non-contagious disease, parasitic disease, metabolic disease, etc. The device is adaptable to different levels of privacy and to multiple scopes of disease detection. In non-clinical settings, the device can be used in search and rescue, e.g., to screen for toxins, and/or to locate injured persons; in military applications, devices may be installed on a vehicle or drone to screen, e.g., for toxins or chemical signals associated with explosives; the device is also available to search for concealed explosives, weapons, persons, etc., either in a handheld or drone held format.

For personal use, perhaps a serviceperson, e.g., an HVAC inspector, entering an establishment is screened in a manner similar to the clinical protocol, whereupon a signal indicates the disease or diseases is/are detected or not. If a disease of interest is detected the serviceperson is denied entry. In such case a message may be given privately to enable the person denied entry to access the disease indication that prevented entry. The device might be set for a particular pandemic disease actively infecting the subject whereupon a rejection is flagged for the one disease. In flu season, the device may be set to detect several influenza strains active in the area and thereby decrease contagious spread between individuals or groups.

To accomplish these tasks, the present invention provides a device and process for assaying volatile organic compounds (VOCs) in a gaseous state bio-sample from a non-invasive bio-sample by collecting gas emitted through a subject's skin into their ambient surroundings. The invention further provides a device that optimizes the capture, classification and pattern recognition necessary for identifying a unique VOC profile or signature derived from that bio-sample. The preferred device features an inhaler ending with an extendable probe that can be appropriately configured with specialized attachments to collect gases from an enclosed area, the air surrounding any person, and, in clinical applications: near any skin surface, from any zone or zones within or immediate adjacent to a body orifice, including, but not limited to: palm, otic canal, nasal passages, mouth, armpit, vagina, urethra, navel, anus, scalp, torso, foot, etc. The probes accessing vapor phase gas are characterized as scopes in the sense that they are used to obtain a metabolic picture of the subject's health status. One or more probes may be specialized for certain sample collection zones, for example, the mouth or throat. One or more multi-purpose scopes may differ in size, length, or diameter. For example a longer scope may permit a clinician to guide the probe from a greater distance from the subject. A smaller diameter may feature a smaller intake port or an adjustable intake port to reduce the size of vapor phase collection area. A small diameter multi-purpose scope may function as a probe external to one of the orifices of interest. For example, otic gas may be collected internally using an otoscope shaped device, but otic gases can also be collected using a multi-purpose scope guided to a zone immediately outside the ear canal. A probe or scope in the present invention may include a visual lens or camera, but such imaging is not a requirement of the probes sometimes referred to as “scopes” in the context of the invention description.

This invention enables quick and accurate analysis for recognition of one or more diseases of special concern simply by collecting and analyzing VOCs emitted through the skin or exhaled into the ambient air. By assaying VOCs in real time, this invention provides a practical screening device for clearing persons of interest, e.g., at a hospital entrance, in an emergency facility, in a waiting room, planning to enter a room or building, a client seeking a personal meeting, etc., and/or providing a handyman, health care worker, or other service provider, a means of assurance when entering a place of service. The device is self-contained, handheld and portable. Thus it can be used indoors, at an entrance way, and/or outdoors in a crowd setting or as a screen before entering a building. Such outdoor screen may be used to direct the subject to the optimal venue for entrance. The device case and electronics may be hardened, e.g., as desired for military, private, confidential, or high stress applications, for severe environments and/or against electronic eavesdropping or interference. Weatherproofing, specifically designed temperature/pressure windows for electronics, decontamination hardiness are optional features in some embodiments. The device can therefore be configured for normal indoor uses, extreme weather conditions (such as desert, high humidity, and polar regions, mountaintops, etc.), conditions of space exploration, moving vehicles, etc.

The invention disclosed herein improves both in the clinic with rapid diagnostic experiences and improved outcomes and for local or personal screening to gain confidence in close interactions. In addition to personal or routine use as a screening tool for persons expecting “prolonged” contact, e.g., as short as about five minutes close contact, fifteen minutes in the same room (as advised times of concern for corona virus exposure) the device is an effective clinical diagnostic tool when configured in a mode to screen for multiple contagious and non-contagious diseases thereby serving as a rapid tool for diagnosis. Those shown to be free of the VOCs constituting the disease or bio-threat “signatures” probed for can be allowed a degree of comfort while those expressing one or more signature patterns associated with a disease or bio-threat can receive prompt information regarding diseases of concern and appropriate direction regarding potential remediation. A “signature” in this context is a composite of VOCs identified in a pattern as being produced by cells of individuals infected by an identified microorganism or associated with a bio-threat. When used in a clinical environment, through early detection and real-time test results, this invention can increase both survivability and quality of life of patients. Costs and maintenance are minimized since the device is self-contained and requires no reagents to be mixed or added during continuous operations.

### BACKGROUND

In the realm of a viral disease, viruses require entry into cells for replication, infection and contagion. Viruses bind to specific receptors on cells and once the viral genetic material is incorporated into a recipient cell's metabolism, the virus exploits the cells metabolism to perform virally related chemical reactions. These reactions are not normal reactions of the recipient cell, but reactions that have been introduced (from the viral genome) or accelerated, reduced or eliminated in response to the viral hijacking. In many viral attacks, an infected cell may recognize that it, the infected cell, is a hazard to its parent organism and will send signals to the parent to request an immune response. Thus, a collection of VOCs, some that may merely be indicative that a viral attack is occurring, while others will be a result of novel synthesis of viral components will manifest.

Accordingly, a “signature” of a disease will not generally rely on a single VOC, but will consider a balance or ratios between several distinct VOCs. The VOCs of interest will generally be associated with the virus itself, but often will include specific information, e.g., of the infected recipient cell, that is cells of a type having a receptor that the spreading virus binds to before entering the cell. At the outset of an infection, when the viral genetic material is first being expressed, VOCs resulting from the virus-induced reaction using the host/recipient cell machinery will manifest, at a stage before immune response is ramped up. Asymptomatic or pre-symptomatic individuals, since the viral replication metabolisms are in progress, therefore can be expected to produce a VOC signature associated with the viral disease even, for example, before the subject presents with elevated temperature, increased white cell count, pneumonia, etc. Understanding that the different viruses and different cells under attack will produce their respective signature VOCs, the device of the present invention can be programmed and/or designed to signal an alert for a single infectious disease of interest or to report results relating to a plurality of diseases or conditions.

The present invention has two fundamental components: the first for collecting, measuring and assaying VOCs in its vicinity, and the second for processing the VOC data into useful output. This compact device can be accessorized with one or more interchangeable probe ends that can be easily targeted towards an area of interest, e.g., a sore throat, the palm of a hand, an armpit, general area surrounding a person, etc. The accessory tip on the probe end collects a sample and delivers it through a tube to an assay module. The gases flowing from the subject through the tube enter the module which rapidly assays the non-invasively or minimally invasively obtained bio-sample, for patterns associated with one or more disease signatures. The assay module intercourses with data processing components to compare the assay data patterns to a library the relevant assay data results expressed as “signatures” associated with or characteristic of preselected diseases. The preselected diseases may include all diseases in a library, e.g., a library of pediatric diseases, a library of infectious diseases, a library of influenzas, a library of oncologic diseases, a library comprising a single preselected disease, a library that includes all available signatures, etc.

The libraries are developed through selecting a group of subjects, e.g., 10, 12, 15, 20 diagnosed with the selected disease. A control group, subjects not diagnosed with the selected disease, provides a comparison sample of assay results. VOC assays from the control group identify non-relevant VOC patterns. Common VOCs from the disease group that are not also common to a control group are combined and characterized to form a draft signature or profile. Pattern recognition is preferably accomplished using machine learning or artificial intelligence. A second disease group is selected and assayed to confirm the earlier signature patterns. As the device is used, additional subjects provide additional results which can be used to refine the recognition and association processes. The process is repeated for the next selected disease.

The device detects and characterizes an extremely large number of VOCs. Data analysis and processing identifies and then ignores VOCs are not involved in disease detection. A sample profile is thereby formulated for comparison with VOCs in the selected library or libraries. The device thus assays for specific patterns of VOCs to identify one or more designated signatures associated with a particular disease or bio-threat. A distinguishing feature of this invention is its ability to rapidly obtain and analyze, in real time, VOC data from its sample of vapor components, rather than from generally more difficult to harvest and manage solids or liquids. The device assay module features durable sensor elements appropriate for mounting within a supporting structure, easily fabricated for durability and rugged use. The probe and connective tubing direct gas for analysis by the sensor elements in the assay module. The assay process using this device requires no special chemical or biological reagents, sample preparation or inter-sample maintenance. In many circumstances only a cursory cleaning or wiping of the sensor tip may be advised before screening the next person. No physical contact between device and subject is necessary when gases are collected from the ambient emissions. In many embodiments the probe tip is replaceable routinely or if compromised or damaged, interchangeable with other specialized probe tips, and is durable to sonication, autoclaving, uv radiation, etc.

Advantageous sites for collecting VOCs include any warm body site (for more robust volatilization), any site with thin skin near active blood vessels, any body zone or body orifice that might advantageously produce, concentrate, or emit VOCs. One such site is the axillary zone, aka, the armpit. The human armpit is known for its ability to produce eccrine and apocrine sweat with resultant odors or vapors. While weaker VOC signals may be obtained proximal to any body surface, the armpit area provides a stable concentration of VOCs thereby standardizing the time for collection and analysis while maintaining sensitivity. Sensing can be from any zone or part of the subject body. Specialized probes can be attached to improve collection from particular body parts or interest. Some probes may include caps that guide and/or maintain positioning or specialized shapes for obtaining a gas sample from a preselected body orifice.

In one application for using the device, a technician, heath care worker, or device operator (clinician) uses the device to monitor gases from the otic canal area of a subject. As an example: the clinician's right hand guides the probe to the proximity of the subject's ear (otic canal); the clinician's left hand holds the device with the handle/trigger portion under the sensor module box. When activated, a suction fan or pump draws gas from the otic canal area into the probe. The otic gases travel through the tubing to the sensor module where the gases pass across the sensing chips for molecular assay. The device contains analytics in the box for processing the data and reporting results such as on a touchscreen on top of the box. The touchscreen may be configured to allow operator input, e.g., selecting the assay type or orifice selected as well as presenting a user interface output to provide results visually in graphic or text format.

The device may rely on natural convection and/or diffusion but speed and volume of collection can be improved when the “scope” (probe at the end of a tube) draws in air to force higher amounts of gas molecules to interface with the nanosensing elements (NSEs). The moving air also can free or dislodge VOCS loosely adhered to skin or clothing. An optional gas selective membrane or filter allows vapors to cross into the analysis collection chamber while preventing interference from, e.g., dust or water droplets. Physical barriers or shields may be used in front and/or behind, e.g., the armpit, to set and maintain position and to reduce dilution from the surrounding air. A less simplified device may feature a warming or heating function that may stimulate volatilization and release of VOCs and augment molecular arrival at the sensors. Some embodiments may incorporate a temperature sensing mechanism or thermometer. This thermometer my suggest that the subject may have a fever and thus is a candidate to receive additional screening(s). The thermometer or thermometers can be used to control the warming or heating function within the collection module and/or also may be used for tuning the sensor elements to maintain the differentiated VOC sensitivities that may present at elevated temperatures. Similarly, some embodiments may incorporate a hygrometer for further refinement in measuring and reporting.

Nano sensors arrayed beyond the membrane or filter are designed to selectively attract or repel a panoply of VOC molecular species. The charge may be static on each NSE or may be varied to include consideration of charge effect on the molecule. An interacting and thus detected molecule as it approaches, arrives in close proximity, and leaves a NSE will change the electrical characteristics of the NSE transiently, if only briefly interacting, or longer if the affinity is greater. Changed charged on the NSE and resultant disengagement of this molecule is one form of useful information that can be incorporated into the profile or signature which may consider multiple scores of such events on different NSEs. The different NSEs may differ on charge provided, may differ on the decorations (functionalizing addends, e.g., short nucleic acids), may be maintained at different temperatures, etc. Charges and/or temperatures of a group of NSEs or any individual NSE may be controlled in accordance with protocols designed for distinguishing VOCs associated within the disease signature profile from VOCs not of interest. Individual or group modulation may be pre-programmed and/or may be adjusted in response to test factors including, but not limited to: gas flow rate, axillary temperature, ambient temperature, humidity, etc.

Data from the NSEs may be analyzed and compiled within the device further analysis and report generation may be contemporaneous or batched processed following transmission (e.g., wirelessly or through a tethered cable) to an external computer or computers.

Thus, the assays obtained using device(s) of this invention initiate with a probe or baton guided to position near a collection zone or area. A cap or shield structure at or near the terminus of the measuring probe may facilitate appropriate placement and act as a shield or barrier to airflow from distant zones. Taking the armpit as an example, a second shield may be positioned at the opposite side of the axilla, both to shield the armpit from the outside air during data collection and to shield the test subject from others, e.g. the device operator. Gases are collected passively or actively within the scope and allowed to pass in close proximity to at least one array of nano-sensor elements. The NSE responses are monitored and recorded for analysis of responses from the various NSEs. The analysis and/or subsequent analyses produces signature data for comparative use in identifying subjects infected by an organism under investigation or a possible unknown bio-threat.

The device may report results by a simple signal, e.g., a tone, a light, a locking of doors, etc. The signal format is not restricted, perhaps a tone or light frequency may divulge important information; e.g., a high beeping tone or flashing red light may signal that the subject should not be allowed to proceed; a soothing tone or green light might indicate passage is approved, etc. More complex signals such as voice commands, lights progressing down a desired path, arrows or physical prodding or gate opening are also potential signals.

A more elegant probe device may feature a coiled tubing structure that may be retracted or self retract when not in active use. The length of the device may be adjusted to allow selective control of the distance between operator and subject. A short wand or rod may be attached to the probe or distal tubing to provide a pointer grip for the operator to effect probe positioning.

After VOC data collection and analysis, the signature information is available to the operator and/or subject and is preferable compiled and further analyzed across multiple subjects to continuously improve or fine tune the devices. The volumes of data obtained suggest that machine learning or artificial intelligence will be involved.

The present invention features a flexible tube connecting a probe to the sensing module of the device. A trigger in a handheld grip controls operation. The grip serves as a handle for the box that houses the sensing module with the NSEs, electronic controls, data capture and processing capacities. When activated, the trigger starts a process to draw air (gas) from the probe end, through the tubing and across NSEs in the sensing module. The grip may be considered a portion of the attached box when it incorporates box components such as a power supply (generally a Lit ion power pack) and may include an exhaust port at its base. An alternative embodiment opens a port after the sensing module to draw air though it, the connected tubing, and the probe. The suction may feed into a control chamber that standardizes the pressure of gas to control the flow rate across the NSEs. The control chamber may be housed in the box per se but its location is not critical to operations. For example, The control chamber may be located in the trigger or in a probe. The chamber pressure may be controlled by a pump to create a higher than ambient pressure. When the port leading to the sensing chips is opened the pressurized gas flows across the chips. Gases may be continually fed by steadily reducing the volume of the control chamber. Or a metering pump may control the flow. The control chamber gases may be heated or cooled for an additional degree of standardization. Sample gases may be delivered in pulses to obtain additional data for normalization.

A preferred embodiment incorporates a screen on the box surface. The screen may indicate settings, progress, results, etc. A touch screen such as on a cell phone or tablet is a preferred user interface. An operator may, for example, enter a tag associating data with a subject, select the type of assay, select which libraries to be queried, etc. Such screen may be in a portrait or landscape orientation. In preferred embodiments, the orientation can be set by the operator. A camera associated with the screen may scan be used to scan machine readable language or code to identify the subject and/or task at hand. The box may itself preferably incorporates the touch screen and com-com features, but in some embodiments may sport a port for accepting such accessory device. Other embodiments feature the box associated with a cradle for connecting a cell phone or similar device. The cradle preferably includes a selection of connectors to access different styles or brands of devices through the cradle connection(s). When associated with a screened device such as a cell phone, the box may use its user interfaces, e.g., touch screen or voice for inputs and outputs.

In certain configurations, the probe may have a plate-like structure opposite the handle. This plate-like structure can be used to achieve optimal placement of the device on the subject for assay and help to isolate body VOCs from the environmental air.

The device may incorporate computation and communication operations by incorporating such hardware and features as common in smartphones. The device may be configured with physical or electromagnetic communication ports to interface through a communication device such as smart phone. The flexible tube may be outfitted from a selection of intakes (probes) with appropriate flexibility, diameter, and lubricosity, including, but not limited to a tubular sponge, an axillary probe, an otic canal probe, a throat swabbing or invading probe, a nasal insertion probe, a groin probe, an anal probe, a cystoscopic probe, a foot probe, a probe for any orifice or targetable body area or zone.

The probe portion connects through the flexible tubing to a box (of any convenient dimension) that preferably aligns with or incorporates a smartphone or smartphone like computational communicator (com-com) device. The device also incorporates a power supply such as a Lit battery or other compact power. Air is hoovered through the sensing module from the tip of the probe to feed it through the tubing to take the collected air to a sensing array or grid in the sensing module.

An accessory kit in some embodiments includes a cradle or sleeve to associate the air collection module with the com-com device. The cradle portion is preferably larger than the com-com to provide a degree of shielding partitioning the com-com from the detection module. The detection module in addition to the electronics involved in the sensing operations contains at least minimal, but adequate, data processing hardware and software that are sufficient to interface with the com-com. The com-com components in the box/screen module may receive accessory data from medical records or contemporary readings from other devices. This module may also update medical files in real time and/or send data to coordinated devices and/or a designated recipient. The recipient may be an authorized database or an authorized individual. When desired the device may transmit to a printer to allow a medical worker, patient or guardian to visualized graphical results and/or text reports. These interconnected data may be associated and stored with the subject profile for reference or may participate in possible fine tuning of patient therapies.

The com-com interfaces may also exchange biometric data, such as obtainable through a finger clip, chest or limb strap. For example, a fingerclip shaped like a pulse-oximeter clip can provide pO₂, heart rate, pulse strength, perfusion index, temperature, etc., to the com-com for association with the VOC assay data. Data transfer and other communications may be through close contact electromagnetic interface, or preferably though a plug-in or hardwired interface. The smartphone like device has software installed or an app available for interfacing with the probe and/or detection module.

Armpit gases from sweat serve as one preferred source for sampling because the apocrine and eccrine glands filter body fluids and secrete water for cooling along with body odors secreted for recognition and other legacies. Thus, information from the entire body is available in sweat.

In one exemplary use, a person being screened (the subject) steps on a marked square, spot, or booth in a clinic. If the axilla is the preselected area, the subject is advised to raise one or both arms. The probe is inserted near the axillary or other preselected area and the subject is asked to lower the arm(s), be seated, leave the booth or otherwise become comfortable. The probe is retracted allowing subject to move freely.

Preferred embodiments of the device feature a thermometer function that measures and reports a subject's body temperature in the enclosed armpit. This may suggest a follow-up relating a possible fever condition in the subject, but an elevated temperature may have resulted from the subject's wait in warm and/or high humidity conditions, high exertion to arrive at the entrance/test station, etc. An elevated temperature alone may not be indicative of a disease condition. Recognizing that temperature affects VOC volatilization, several embodiments revise the standard test protocol, e.g., to initiate a warming stage, to set voltages and temperatures on the NSEs, etc.

For rapid screening a subject may be advised of a positive result at this stage and advised to seek treatment. Since in many instances the screening device will only contact clothing, not the subject, procedural cleaning protocols are optional. For assays requiring or risking contact between probe and subject, a sleeve

over the probe may used. Such sleeve is designed with its end protecting the probe from contact while allowing gases to port through the probe into the tube. A probe can be constructed of durable composition allowing chemical, radiation and/or heat sterilization. Since the probe itself has no electronic or mechanically controlled parts, the cost is low and may be considered expendable. In several embodiments the probe length is surrounded by a perforated or porous, e.g., open sheath or foam padding which is designed to be washable or disposable.

The present invention especially features a collection probe attachments suitable in size and lubricity for monitoring gases harvested from human or animal orifices. The sinuses or the otic canals allow free collection of gases. In moistened orifices, an assay may be timed to account for diffusion of gas from the liquid partition into the gas partition of the probe. However collected the gases are measured and analyzed to form a subject profile for comparison against signatures in one or more selected disease libraries.

Signatures are not limited to any single VOC, class of VOCs, select groupings of VOCs, amalgamations of cross reacting VOCs, etc. And may be analyzed in one or more algorithms involving non-VOC and/or alternatively sensed or obtained data. Such data may include but is not limited to genetic data, age, gender, health history, ambient or internal non-VOC presence or concentration (e.g., circulating, tissue, ambient gas, temperature, season, blood work, stool work, urine sample, skin sample, hair sample, other bio-sample (e.g., saliva, spit, tear mucus, time of day, light level, travel history, etc., and in the case of the present invention: sweat, apocrine gland secretion, eccrine gland secretion, sebaceous gland secretion, axillary microbiome secretion or content). Examples of VOCs and or other relevant compounds that are available for analysis include, but are not limited to: androstenol., dehydroepiandrosterone, dehydroepiandrosterone sulfate, androsterone, andosterone sulfate, lactic acid, acetic acid, isovoleric acid, cholesterol, 5-androst-16-en-3-ol, aniline, o-toluidine dodecanol, progesterone, one or several ketones, one or more fatty acids, one or more fatty esters, one or more alcohols, ethanol, hormones, steroids, endogenous cannabinoids, styrene, naphthalene, benzaldehyde, tetrachloroethylene, propanol, diphenylamine, butanol, pentanol, H₂S, CH₃SH, (CH₃)₂, dodecanol, tetradecanol, 2-ethylhexanol, phenol, p-cresol, 2-methylaniline, pyridine, 3-hydroxy-2-butanone, propionic acid, iso- and n-butyric acids, phenylacetaldehyde, furfuryl alcohol, isovaleric acid, α-methylbutyric acid, dimethylsulfone, n-dodecanol, n-hexadecanol, p-cresol, indole, benzaldehyde, benzoic acid, ethylene glycol, propylene glycol, propanoic acid, n-butyric acid, hydroxy-ketones, isobutyric acid, 3-hydroxy-2-butanone, 2-methylbutyric acid, 2-hydroxypropanone, valeric acid, myristic acid, palmitic acid, phenylacetaldehyde, palmitoleic acid, steric acid, oleic acid and urea.

As an analogous example, compounds that have been measured in breath and/or surrounding ambiance include, but are not limited to: butane, 3-methyl tridecane, 7-methyl tridecane, 4-methyl octane, 3-methyl hexane, heptane, 2-methyl hexane, pentane, 5-methyl decane, IL-6, endothelin-1, methylated-including monomethylated—and other branched alkanes, acetaldehyde, formaldehyde, 2-methylpropanal, 3-methylbutanal, 2-methylbutanal, butyl acetate, 3-heptanone, 2-amino-5-isopropyl-8-methyl-1-azulenecar-bonitrile, 6-ethyl-3-octyl ester-2-trifluoromethylbenzoic acid, 2,3,4,6-tetramethoxystyrene, 2,4,6-tris(1-methylethyl)-phenol, 1,3,5-cycloheptatriene, and 2-methoxy-acetate ethanol, butylated hydroxytoluene, 1-methyl-3-(1-methylethyl)-benzene, and 4,6-di(1,1-dimethyl-ethyl)-2-methyl-phenol, 2-amino-5-isopropyl-8-methyl-1-azulenecarbonitrile, 2,2-dimethyl-decane, carbonic dihydrazide, 4,6-di(1,1-dimethylethyl)-2-methyl-phenol, butylated hydroxytoluene, 3,3-dimethyl-pentane, 5-(2-methylpropyl)-nonane, 2,3,4-trimethyl-decane, 2,2,4,4,6,8,8-heptamethyl-nonane, ethyl benzene, 2,2,4,4,5,5,7,7-octamethyloctane, hydroxymethyl 2-hydroxy-2-methylpropionate, 2-methyl-hexane, etc.

The assays forming the profiles and signatures used in this invention, have no requirement to identify the chemical formula or structure of VOCs in the identified patterns. The sensor module is sensitive to molecular movement. The characteristic movement behavior of a molecule in its proximal interactions with each single NSE-compound interaction, i.e., the behavior of a molecule, not its structure forms the patterns. Out put may be simply a list of positive and negative test comparisons, that may include guidance relating to test experience, e.g., “This identification has a recognized specificity in relation to disease xxx of 92-96%.” A graphical representation of the VOCs may be displayed, stored and/or printed if desired.

A chemical breakdown of the VOCs may be made available in a report, for example a detailed report that includes intensity of each relevant component. Suspect known VOCs may be assayed to confirm the match to the movement behavior data. Analytical chemistry in conjunction with identification of the disease associated VOCs may help understand the disease processes. The knowledge of specific relevant VOCs may lead to improved sensor programming, decorations, or other selective modifications in the assay device or program.

Historical data may be collected from a single patient to provide a timeline comparison, e.g., to determine if a particular course of treatment is working or has been effective in reducing the signature VOCs. Similarly, for prolonged care, the progression of, for example, a cancerous disease or autoimmune syndrome may be plotted and followed using the acquired data.

### Advanced Analytics

To date laborious techniques such as MS/GC have been the preferred analytical tool for VOC analysis. Improvements in detection sensitivity from micro-detection to nano-detection using highly advanced sensors now enables a more robust use of nano-analysis of VOCs and other compounds and when combined with rapid data analysis and machine learning can: a) confirm a diagnosis, b) assist in selecting or ranking diagnoses and/or c) suggest one or more diagnoses even prior to outward symptoms becoming apparent. After assay, in some circumstances simply questioning a patient about a result may elucidate an overlooked symptom of disease.

The general approach of monitoring VOCs for detecting disease has been in development for several decades and now is soundly acknowledged in developing science and health medicine. In accord with the present invention a device to achieve these VOC assay goals has been designed to monitor behavior of a variety of volatile organic compounds (VOCs) in a rapid and reproducible fashion. Under the present invention, multiple disease signatures can now simultaneously be searched using a single sample. The basic benefits of measuring VOCs for disease detection have been recognized in medicine for quite some time. In a 2014 Clinical Policy Bulletin, Aetna explained its policy regarding VOC analysis at that time as:

Aetna considers the analysis of volatile organic compounds experimental and investigational for the following indications (not an all-inclusive list) because the clinical effectiveness of this technique has not been established:
Detection of bacteriuria
Detection of cancer (e.g., breast cancer, colorectal cancer, lung cancer and cancer of the pleura, pancreatic cancer; not an all-inclusive list)
Diagnosis of amyotrophic lateral sclerosis
Diagnosis of autism spectrum disorders
Diagnosis of inflammatory bowel disease
Diagnosis of juvenile idiopathic arthritis
Diagnosis of non-alcoholic fatty liver disease
Differential diagnosis of breast diseases (e.g., breast cancer, cyclomastopathy, and mammary gland fibroma)
Prediction of development of childhood obesity
Use as markers for monitoring hemodialysis efficiencyl ¹ Clinical Policy Bulletin: Analysis of Volatile Organic Compounds. Number: 0717. Revised April 2014. Obtained from: http://qawww.aetna.com/cpb/medical/data/700_799/0717_draft.html

### Advances in VOC Technology

Urine, exhaled breath, and blood are classically recognized as available sample sources. The present invention features assays of vapors, e.g., breath, sweat by-product, otic gas, etc., as preferred sources for the assays.

A device, as described herein, capable of providing a sample profile to match against signature information from previous identified assays, including bioassays and/or assays of off=gases from structures suspected of emitting possible harmful compounds into ambient atmosphere, meets multiple identified needs and applications.

The device of the present invention provides rapid highly sensitive detection of VOCs in a gas phase sample, e.g., vapors collected by the device held in or near the armpit, in or near the otic canal, near a palm or forearm, etc. Analytical data are then processed using the device's dedicated or developing algorithms to detect a disease or to answer questions for which the sample was taken. Through machine learning and artificial intelligence, the device is continually developing and improving its algorithms.

Through capturing the VOCs in vapor or gas phase to measure the presence, amounts, volume, intensity or strength of signal of each of multiple VOCs, then classifying each signal as from the organism or the environment and removing foreign or control VOCs from analysis consideration, the device then outputs a report profiling a sample's gross output of organism initiated organic compounds for comparison to the signature database to determine whether a specific disease (or set of diseases) is present. The present invention in a preferred world continues to consolidate VOC signature profiles into a library as new sample outputs are presented.

In addition, the device may physically incorporate add-on devices and/or applications, for example, a capillary analytical attachment, including, but not limited to: capillary electrophoresis, capillary chromatography, capillary ELISA, nano-sensors similar to the vapor phase sensors but proximal to analyte in a liquid phase, etc. Add-on devices may be analytical providing additional information to be used in data analysis and signature identification or in some embodiments may absorb, adsorb, catalytically modify and/or filter out potential confounding compounds and thereby minimize the necessity for applying algorithms to remove the undesired ambient VOCs. The machine learning component of the invention, in preferred embodiments, has capacity for inclusion of externally generated information from add-on devices and/or from externally provided information.

One preferred format of the present invention features “chips” with modular nano-sensing elements (or nano-sensor element (NSE) that are independently maintained at a fixed, fluctuating, stochastic, alternating, discontinuous or flashing feeder power supply. Chips may be provided in a cartridge format allowing easy interchangeability. All replaceable components are designed to be returnable and/or recyclable. The outputs of each NSE may be individually wired to a dedicated data transducer or a selection or grouping of sensor outputs may use a common carrier circuit and thus be “averaged”. In some embodiments, a simpler circuitry may involve multiple elements feeding a single output that may sum the outputs to deliver an average reading. When one or more of the “averaged” sensors is turned off or powered down, the average will not include output from these one or more powered down sensors. When input sensors are powered individually, for example, in a cycling pattern when only one (or a selected portion) of the input electrodes is being charged, averaged outputs synchronized with the timing of input charging can thus provide data from individual channels.

Sensors may act independently and/or in concert: in parallel or in series. During development, signatures may be derived from multiple sites including, but not limited to: ambient environment, body secretion (internal or external), natural orifice, artificial orifice, etc.

The single output may connect and thereby collect data signal from any desired fraction of sensing elements. For example, a single output may receive signal from all elements on a chip, half the elements on a chip, one-third the elements on a chip, a quarter the elements on a chip, a fifth the elements on a chip, and so on, for example, 1/6, 1/7, 1/8/, 1/9, 1/10, 1/12, 1/20, 1/25, 1/33, 1/50, 1/100, etc. Any output may be associated with 1, 2, 3, 4, 5, 6, 7, 8, 9, 10, 11, 12, 13, 14, 15, 16, 17, 18, 19, 20, 21, 22, . . . , 24, . . . , 32, . . . ,48, . . . , 50, . . . , 64, . . . , 96, . . . , 100, . . . , 128, . . . , 200, . . . , 250, . . . , 256, . . . , 500, . . . , 512, . . . , 1000, . . . , 1024, 2048, . . . , 4096, . . . , 5000, -8192, . . . , 10,000 (104), 16, 384, . . . , 2¹⁵, . . . , 2¹⁶, . . . , 10⁵, . . . , 2¹⁷, . . . , 2¹⁸, . . . , 2¹⁹, . . . , 10⁶, . . . , 2²⁰, . . . , total number of sensors on a chip, which may vary with time or programmed instructions. The precise count of sensor elements associated with any output, in general, is a design feature and does not define operative functions of the invention. The counts specifically exemplified above are exemplary low numbers of sensors that may feed an output and higher numbers common in conventional plate assays and powers of 2 and 10 frequently used or approximated in biological or chemical science or physics or electronics.

When connected to multiple elements, the output averaging output signals from each, connection of each element is optionally modulated to alter weightings of each of the elements in the average. With fluctuating or non-constant inputs weighting is also controllable. For example, in an extreme sense a stochastic or alternating input, when alternated to off that element's output will report a zero weighting, or a fluctuating or stochastic feed can serve to physically, rather than mathematically control the weighting output. The designer and/or operator will have options for mathematical/algorithmic or physical/electrical weighting of each NSE input to the data analysis. A group of elements may therefore receive the same feeder voltage, or the feeders may be independently controlled.

Instruction to, or control of, the system may be through information encoded on a sample probe package, information encoded on a sensor chip, from a user interface, information provided remotely by machine or active user, or information encoded within the device. For example, during field work or development mode, samples may be assayed, then stored for possible later analysis. Labels may be encoded using any conventional or available tagging.

When the sensing probe is positioned for assay, the operator can manually initiate the collection process, e.g., using a triggered handle. The operator may visually and/or tactilely determine that the sensing device is in position; may ask the subject if the device is comfortably disposed-if contact between subject and device is involved; may withdraw the probe after a predetermined time or following a termination signal from the device; and/or receive feedback from the device to indicate that repositioning is advised. The device may inform the operator of conditions by any suitable means, e.g., light, sound, vibration, movement of a switch or switch guard to a ready position. A vibration or similar tactile signal may be especially useful in crowded, noisy venues. In some embodiments, after positioning of the device in proximity to the target zone, initiation may be triggered temporally, mechanically or automatically, e.g., using light, tactile sensors, and/or temperature sensors.

The collection process may include a warming or heating stage in conjunction with or as a prelude to sucking gas through to the probe and into the sensing module. Gentle heating inside an orifice can be through any conventional means. In non-contact gas harvesting, heat may be provided using natural or artificial stimulation, e.g., light absorbed for heating skin in a range from approximately 610 to 900 nm is effective. Such heat may favorably facilitate release of VOCs for analysis.

Though the flow through the device is essentially continuous, the flow rates over the NSEs or driving VOCs towards the NSEs may be pulsed, i.e., varying in regular (e.g., continuous waveform similar to a sine wave, spikes, etc.) or irregular (e.g., asymmetric, perhaps a ramp or wave upstroke with a different shape downstroke including a vertical drop). The pulsing continuing flow may feature a flash start of a strong pulsed flow with a ramped or declining wave to continue flow but preparing for the next pulse. The flow may be modulated or controlled using an elevated pressure, a fan, a piston pump, a volume displacement pump, a peristaltic pump, or the like.

During sensing, a meter (visual, sonic, photonic, etc.) may be actuated to indicate progress. For example an analogue styled meter may have colored zones in a bar or pie shape format to light when the subject's gas is flowing or being detected with colored zones or separate bars to indicate time of or time to completion of one or more stages, e.g., a heating stage, a collection stage, an analysis stage, etc. Rather than or in addition to time, the display may indicate temperature, volume or a count of VOC molecules crossing the sensor pads. Sound or light cues can be provided at various evens in the assay. For example, a blue light may indicate the rod is properly situated and ready to begin. The “begin” may relate to initiation of the collection-analysis process that in some embodiments includes a warming phase. The color may change as different phases are actuated, e.g., a color for ready, a color for warming, a color for collecting, a color for completion. At completion different colors can indicate different outcomes. For example, green to indicate the subject is not a risk; red to indicate a risk of contagion.

### Organization of NSEs

The sensor module preferably features nano-sensor elements (NSEs) to minimize size and maximize sensitivity of the sensing chip. NSEs will in general be mounted or carried on a substrate or support matrix forming a “chip”. Individual matrices may feature multiple elements, generally 10 or more, 32 or more, 50 or more, or larger populations of elements on a single chip. As a rule, a greater number on a chip promotes a compactness desired for minimizing weight and size. The number of NSEs is a design feature and can mimic numbers familiar to the operator or data analyst. For example, multiples of the number of wells common on petri dishes may facilitate using existing software tools to further analyze and compare results. Powers of ten, multiples of a hundred or thousand, powers of two are in common use. Accordingly, about 96 elements, 100 elements, 128 elements, 144 elements, 200 or 256 elements, 500 or 512 elements, 10³ or 2¹⁰ elements, 10⁴, 2²⁰, 10⁶, etc. May be built in as common useful working populations even if several elements on the chip are not activated.

Minor variances in sensor sensitivities may be weighted internally by the machine software or may be overcome by averaging signals of a subpopulation of elements on a chip or chips. This massaging feature is preferably available and used as a tool to promote inter-device and/or inter-chip consistency.

NSEs carried on the chips can be any properly designed sensing surface capable of, for example, field-effect transistor (FET) or other physico-electrical property/activity including, but not limited to: semi-conducting nano-wires, carbon nano-tubes-including single-wall carbon nano-tubes, chitosan-cantilever based, synthetic polymers-including dendrimers, plasmon resonance nano-sensors, Förster resonance energy transfer nano-sensors, vibrational phonon nano-sensors, optical emitting, optical frequency (or wavelength) based nano-sensors (sensitive to photon transmittance, absorption, reflection, energy modulation, etc.). Nano FETs and other nano-sensor formats generally operate by changing electrical properties as a substance comes in close proximity to the sensor by perturbing the steady state (absent the proximal substance) charges and movements (distribution of electrons) within the nano-sensor. When the transistor effective electrical properties cause an observable change in electron flow (current) this manifestation is one example of sensor competence. The altered distribution of electrons, depending on the design of the nano-sensor, changes one or more electrical properties, e.g., impedance, resistance-conductivity, capacitance, inductance, etc. And thus the physical movement of a detectable particle, e.g., an electron, a photon, etc.

The discussion in this disclosure of the present invention primarily features nano-sensors whose characteristics change depending on association (close proximity with) a chemical substance. Sensation may involve more than one event. For example, in one format of nano-sensor the proximity event may dampen a molecular vibration that is sensed by observing a changed electrical property. Similarly, an optical property, e.g., reflectance, transmittance, refractive index, can be perturbed by proximity to a substance, altering electron distribution within the sensor enough to cause optically detectable geometric changes. The optically related detection format for a nano-sensor may be observed at a specific frequency or range of frequencies, for example moving peak transmittance to another frequency.

Nano-sensors are classified in different ways, for example, the feature being assayed, e.g., movement, temperature, frequency, chemical, current, voltage, etc.; or output, e.g., fluorescence, light, electric property, etc. As an example, fluorescence outputting nano-sensor may be carbon based, e.g., single walled carbon nanotube, graphene, quantum dot based, nucleic acid (RNA, DNA), peptide based, organic polymer based, etc. Photo-acoustic, plasmonic, magnetic, etc. Perturbations are also useful as biosensors and may be applied in features of the present invention.

One format extensively described as an example herein involves use of carbon-based structures having properties of or similar to decorated single wall nano-tubules (SWNTs). The carbon component atoms of the nano-tubules are receptive to complexing with ringed chemical structures (decorations or functionalizations), often accomplished through a non-covalent n-bonding effect. Graphene, having similar single layer carbon geometry, with proper decoration, can also serve as a sensing surface. Evidence indicates the curved carbon structures of the SWNTs demonstrate more consistent FET properties in many use environments with various functionalization (decoration).

Therefore, curved graphene, possibly formed into a corrugated or spiral geometry, (See, e.g., Michael Taeyoung Hwang, et al., Ultrasensitive detection of nucleic acids using deformed graphene channel field effect biosensors. Nat Commun 11, 1543 (2020). https://doi.org/10.1038/s41467-020-15330-9) may demonstrate more promising specificity, speed of analysis, and/or sensitivity over planar graphene for particular applications. As nanotechnology continues to progress additional sensor formats such as those emitting light, electro-conducting polymers, electro-conducting bio-polymers, etc. May become accepted in the art. Embodiments of the present invention may incorporate these improved sensors as their reliability is established. The skilled artisan will generally choose which form of sensor is optimal for performance and cost.

In addition to the field effect electrical sensing set forth as a preferred embodiment, other qualities of thin carbon based used for sensing are possible. Optical, electrochemical or electrical features have been employed with graphene-based biosensors. Forms of graphene have been successfully tested for electrochemical (amperometric, voltammetric, impedimetric, or combinations thereof) and electrical sensing applications. Selected formats have the high electron transfer rate, the high charge-carrier mobility and manageable electrical noise that is necessary for sensitive detection of biomarkers and other biological analytes. Successful assays have been reported in both serum and blood extracts. Optical transparency of graphene monolayers allows use in sensors such as optical-based G-biosensors.

The NSEs themselves or at least portions of the device surrounding the chips are preferably surrounded by a controlled gaseous atmosphere, e.g., air or a compressed gas, generally slightly above ambient pressure when the gases are used to maintain inflation and shape of the sensing module. The sensing chamber itself may have a reduced pressure with respect to the sample introduction area helping to draw VOC gases into contact with the NSEs. A positive device pressure at least one level surrounding the assay chamber is generally preferred to minimize contaminating inflows. The actual pressure where sensing is accomplished however can be varied. VOCs may be delivered by having a negative relative pressure in the chip area with respect to a sample containment or introduction area to cause drawing in sample off-gas when the off-gas collection volume and the sensing volume become connected. VOCs themselves, for example, when heated may produce the pressure difference to drive delivery to the sensor volume.

Since physical delivery or movement is required to bring a candidate compound in contact with an NSE, a physical intervention is often required. Physical movement can be induced as desired by any appropriate force. Forces may be constant, variable, stepped or pulsed, etc. Multiple forces may be used in series or parallel for sample delivery or a single force may be selected from the device's repertoire to enhance delivery and detection of the sample to the NSE(s). In many embodiments, physical movement is provided by convective currents from a high to low pressure across a chip or chips.

In one example, temperature can control speed and movement of target compounds and ambient gases driving the sample compounds to the sensor; pressure difference induces a convective movement. Pressured gas canisters may provide the driving force when mixed with sample gas. Other forces including, but not limited to: electric, magnetic, electromagnetic, acoustic, photo-excitation or photon momentum, etc., may be selected depending on particular circumstance. Forces may be described in a number of ways. For example, a decrease in temperature may induce a relative vacuum thereby creating a convective force. An acoustic force, for example, having one or more oscillating frequencies in a range perhaps between 10 mHz and 100 MHz will often exhibit one or more harmonics (or multiple frequencies). Echoes may result in one or more frequencies that are distinct from the feeder frequency. Geometry and chemical composition of the device may accentuate or dampen frequencies. The acoustic engineer will take into account the importance of such effects when designing the device. A facility for heating the wand may be used to stimulate armpit secretions.

The probe may be set apart from ambiance or surrounded by a gas porous covering. Such covering will allow gases including VOCs to move from the skin to the sensors. Such gas permeable or porous cover may be perforated open celled plastic material, open celled foam a porous coating, random, stitched, woven, etc. The coating may be selected to absorb out common or problematic molecules to simplify the assay stage. A flushing gas may be used to sweep the skin or clothing and free VOCs for assay. Such gas may be generated chemically, may be heat driven, may be mechanically pulsed, may be provided from a pressurized or compressed gas reservoir, etc. Flushing gas may be alternated with a depressurized stage or a relative vacuum where a small amount of gas is introduced to motivate VOCs followed by a small vacuum to draw the VOCs for NSE analysis. In other embodiments pressurized and vacuum processes are coincident with the offset space forcing movement of gases to collect the VOCs.

The gaseous environment in the present invention is an improvement over prior applications of FET sensing in that the response is both quicker and reversible. Reversibility is critical for high-throughput commercial applications in that it allows for the rapid turnover of samples through avoidance of disassembly and/or cleaning between sample readings. The NSEs on a chip are thus available to assay hundreds or thousands of samples in a day. Though subject accessibility is expected to be many fold lower. Reversibility of VOC-NSE attraction can be accomplished simply by increasing the temperature. Flushing with the ambient gas or another gas can also be used. Continuing to monitor the output signaling from the NSEs provides assurance that the sample has been reversibly cleared and the device is in a mode to accept the next sample.

At least one, but often a plurality of sensor chips, may be included in a device. Cartridges may swap out all the chips or a selection of chips in a device. During use, the sensor chips will be mounted in a controlled atmosphere chamber where vapor phase analyte will be introduced to contact with the sensor chips and thus the NSEs. During analysis, input and output voltages are provided and monitored, respectively, as analyte is delivered to the ambient volume over the chip. Only a vapor phase analyte contacts the NSEs. This provides advantages over many liquid phase SWNT and similar sensors in that sensor size can be reduced without having to account for surface tension, liquid phase excipients are not necessary and turnover rate is not compromised by the requirement to remove the liquid carrier.

### Sample Suitability

For medical applications, the analyte sample is most preferably a non-invasive, readily available, biopsied sample including, but not limited to: breath, flatulence, otic canal gas, axillary emissions, ambient air, etc., may be collected. The human armpit and otic canal are suggested collection sites owing robust circulation and excreting glands in the armpit and the concentration of body gases, especially gases from the head in the otic canal. Other preferred sites include sites near an injury to detect localized infection, during surgery to monitor for possible general or localized anoxia, responses to blood pressure drop, stress induced responses, etc.

The device may include one or more modules to stimulate secretion, e.g., by providing a warming or heating effect in the collection zone. For example, the device may be warmed before introduction and puff warmed air over the collection site or may provide a chemical or electrical heating feature to excite secretions. The chemical and/or electrical heating may have an effect of producing a gas to facilitate carrying and delivery of the VOCs for testing. Facilitating gas might also be delivered or provided through a gas delivery tube or pipe; may be sourced from a compressed gas canister; may be inert; may be selected for its interactions with VOCs of interest to improved desired deliveries; etc.

For rapid screening, the axilla is an available source for sampling as the apocrine and eccrine glands filter body fluids and secrete water for cooling and odors for recognition and other legacies. These sweat glands generously release metabolic by-products into the armpit area. Thus, information from the entire body is available in sweat.

The device can be configured in several formats. It may be configured to accept probes of a variety of shapes, including, but not limited to: a wand-like device, a pen or pointer shape, a bulbous shape, a flat—e.g., tongue depressor shaped probe. The device may incorporate additional sensors, e.g., a thermometer, an accelerometer, a laser or other distance indicator, etc. . . .

### Operation of the Device

Depending on delivery method and time of residence in the assay chamber, a sampling rate in the order of several seconds is currently achievable. Sampling rates of about 5/min (10 to 12 seconds per assay) are possible using the device, but may be reduced in practice by subject accessibility.

### Sensor Elements on the Chip

A sensor chip may be formed on any suitable solid or semi-solid surface, rigid or flexible, including, but not limited to: silicone, synthetic polymers, mu-metal, alumina, alkaline earth silicates, ceramics, etc. In any desired configuration. Sensor geometries are selected as desired for, example, a 16×16 sensor array on a chip can provide a compact yet exuberant surface with 256 distinct sensors; a 4×32 sensor array provides 128 distinct sensors on the chip. Chips may be stacked in multilevel arrangements. A pre-amp assigned to each NSE may be used to adjust sensitivity and selectivity. Post amp may modulate emphasis given to am NSE signal. The pre-amp and post amp may also be involved in calibrating the device.

Each of the sensors may be configured to optimally detect a single compound or a class of compounds. The functionalization molecules may be different on each chip to adjust specificity and sensitivity. Charge on the chip and temperature are other means of individualizing chip capabilities. During a single sampling session, a charge and/or temperature may be modulated to expand the repertoire of the sampling chip. A collection of sensors may be arrayed on a chip in any desirable configuration, e.g., for electronic efficiency, assembly preferences, etc., and/or to align with geometries of the sampling surface or sampling device body.

For example, arrays may be constructed to align with squares or powers of 2 as is common in computation devices and some biological plates. Thus, for some embodiments a 2×2 sensor chip may be sufficient. But more often a greater number of chips will be employed for additional sensitivity and discrimination abilities allowing assay results to be collected on a greater number of analyte chemicals. Thus, a 3×3, 4×4, 5×5, 6×6, 8×8, 10×10, 12×12, 15×15, 16×16, 18×18, 20×20, 25×25, and so on, including intervening squares, mentioned and envisioned here, but not exhaustively incorporated in the text format might be constructed. Other non-square formats are also envisioned. In biology plate sizes based on a power of 2 times 3 are often employed. Thus 48 well, 96, well plates, etc. Are common and easily handled by modular software applications. Since binary electronic electronics often increase capacity according to powers of 2, but physical dimensions may not always be supportive of such doubling with each improved version. Software may often be capable of addressing a number in excess of the NSEs on a chip. For example, computations relating to 2⁶ may be used with a 7×7, 8×7, or 50 element chip. A 10×10, i.e., a one-hundred element chip may be served by an application designed for up to 2⁷ (128) element channels. Higher element chips may thus suggest using applications that have capacity for 2⁸, 2⁹, 2¹⁰, 2¹¹, 2¹², 2¹³, 2¹⁴, 2¹⁵, 2¹⁶, 2¹⁷, 2¹⁸, 2¹⁹, 2²⁰, etc., NSE channels. The chip may be configured with one dimension far in excess of the other.

Then NSEs are preferably extremely compact in size (hence designation as “nano-sensing elements”) to permit high density and smaller device footprint. NSEs on a chip will be separated from neighbors by insulation barricades. Many insulators are known which can be selected during design based on parameters such as appearance, size, cost, assured availability, etc. Polyamides are common inexpensive insulation barricades. Circuit board material (e.g., FR-4, CEM-1, CEM-3, RF-35, halocarbons, fluorocarbons, Teflon®, PTFE, polyimide, etc.)

Depending on material and anticipated voltages, an inter-element separation of ˜50 nano-meters (nm) is often sufficient. Larger voltages may require greater isolation distance. The elements themselves may be any desired shape, e.g., rectangular, rhomboid, hexagonal, triangular, elliptical, circular, irregular, crumpled, creviced, shredded, perforated, layered, masked, etc. Sizes can be miniature, e.g., ˜40-50 nm thus suggesting the term nano-sensor. Size is a simple design consideration involving, e.g., manufacturing efficiency, device dimensions, density of sensors, surface to volume ratio of NSE, sensitivity of detection, durability, cost, etc. Accordingly, sizes of elements may be in the area of for example, 40 nm, 50 nm, 75 nm, 100 nm, 200 nm, 250 nm, 500 nm, 0.5 μm, 1 μm, 5 μm, 10 μm, 50 μm, 100 μm. Shapes may be planar, essentially flat on the substrate surface, or at an angle disposed off the surface. Shapes may be irregular, e.g., crumpled or creviced. Shapes may be regular, e.g., hexagonal, creviced, etc. A sensor element disposed on and above a substrate surface may be designed as such to increase or maximize surface area to interact with the vapor that may or may not include a VOC of interest to said sensor.

Although larger sized elements do not cease to function, the advantages of smaller nano-size generally outweigh advantages attributed to larger element size.

The elements will generally be supported on a non-conductive substrate like Si or other insulator including, but not limited to: FR-4, CEM-1, CEM-3, RF-35, halocarbons, fluorocarbons, Teflon®, PTFE, polyimide, etc.

VOCs in Contact with the Chip Sensor Elements

As designed, the NSEs themselves do not contact the subject, only vapor or gaseous phase emissions off the skin or other subject zone are delivered for contact. Vapor constituents, as the vapor flushes through the sampling chamber, will specifically transiently bind to one or more of the decorations on the NSEs. Depending on temperature and possible gas or combination of gases used as ambient atmosphere, the collated NSE data produce a characteristic response for a specific VOC, combination of VOCs, or class of VOCs. A chip generally will be coated with several types of sensor elements whose sensing specificities are distinguished by having different decorations such as nucleic acids attached on their surface. Polyaromatics and aromatic peptides, including synthetics, may also be valuable decorations. Differential specificity of a chip element may be exhibited at different temperatures, in different atmospheres and/or in different sequence patterns of exposure.

Temperature is significant for at least three important reasons. One: at higher temperatures, the molecules will have higher kinetic energy and thus be less likely to sit docilely on a surface. Different VOC compounds will exhibit different specific temperature effects as will different decorations. Changing the temperature during a sampling session can change specificities and sensitivities. Two: the actual VOC chemical may tautomerize or morph, perhaps with a higher temperature favoring a different bonding structure than a lower temperature. A compound might then, in some embodiments, be detected on different NSEs at different temperatures. Such assay response may be used to more specifically identify and assay a particular compound. Multiple chips or sensors in a sensor array may be maintained at different temperatures. Three: improper temperature management can comprise sample integrity, for example, leading to clumping, particulate contamination or other sample degradation. The signature(s) obtained from these analyses may relate to temperature and output signal modulation(s).

In general, the interactions between the NSEs and the sensed analyte are low energy bonds or coordination complexes between organic molecules. Bonds do not involve covalent reactions and thus are reversible by changing the conditions in the chamber. Dilution, i.e., simply flushing with a clean gas, in many circumstances will ready the sensor element for its next round. Optionally, a clean or inert gas is used for flushing and/or a higher temperature is used to decrease interaction between the NSEs and the VOCs. In addition to thermal or convective restoration processes, any format used to physically move the compounds can be used alone or in concert with others to excite/remove the panoply of complexed VOCs. Forces including, but not limited to those of: photo momentum, acoustic, convective gas flow, magnetic, electromagnet, electric, Lorentz force, chip replacement, etc., effects can be used to prepare for a subsequent sample read. Non-convective restorative forces will be especially advantageous in low pressure, or no added gas embodiments. The broad spectrum of restoration options enables testing of multiple samples types in high throughput operations.

### EXAMPLES

On a chip, a simple configuration in binary format comprises an element grid arranged in a 16×16 (2⁴×2⁴) pattern, I.e., 216 (2⁸) elements. Larger chips generally but not necessarily may follow a continuing binary pattern, I.e., 32×32 (1024 or 2¹⁰), 64×64 (4096 or 2¹²), 128×128 (16,384 or 2¹⁴), 256×256 (65,536 or 2¹⁶), etc. A chip may not use all elements as active NSEs. Some may be inactive, some may be held in reserve, some may serve as controls or calibration elements, etc. The chips are functionalized or “decorated” single wall nano-tubules (SWNTs). Nucleic acid molecules are inexpensive decorations that can be made with thousands of options. Using non-natural, i.e., nucleic acids not in the human genome or RNA repertoire, many more specificities can be addressed. Amino acids with ringed structures can be incorporated as functional coordinating binders. Thus, specificities of NSEs are tuned to the desired conditions. Sometimes the identical decoration will display different specificity as temperatures change.

A base voltage of generally is in a relatively low, i.e., non-arcing or insulator damaging range for example around 1 pV, but more normally up to 20V, is applied to an input electrode of a sensing element. 10⁻¹⁸ amp appears as a minimum amount sensitivity with 0.4 fA being characteristic of our current implementation. The voltage may be static or oscillate (either deterministically or stochastically). Oscillation may include ranging from positive to negative voltages, may include simple on-off switching or other square wave pattern, saw tooth pattern, triangle pattern, stochastic, etc. Voltages may be stepped through a range or introduced in a ramping or cyclic (e.g., sinusoidal) pattern or stochastic perturbation. Voltage may be sent to each sensing element individually or the same voltage may be applied to several sensors, including circumstances where all sensors on a chip or array are fed identical input.

In the NSE, current is, or is not, delivered from an input electrode to a corresponding output electrode through e.g., a field effect transistor carbon layer. In one set of examples the carbon layer is formed as a single walled carbon nanotube (SWNT) layer. In the on mode, the SWNT carbon conducts a current through to an output electrode. When the field effect transistor is in the off mode, the current does not conduct. Several such elements are attached to form a nano-sensor chip. The conductance of the SWNTs on the elemental surface is perturbed by close association with a target compound, for example a volatile organic compound. Binding of such target compound modifies conductance of the SWNTs in such a fashion that the coordination binding acts as a transistor switch turning the gate on or off. In some instances, the coordination will be probabilistic with rapid gating as different portions of the target compound may bind to the SWNT, perhaps at slightly different coordinating atoms. Such probabilistic binding may be temperature or voltage dependent or may vary with the delivering gas. In other instances, the binding may be more constant, simply gating for a range of temperatures/voltages with large zones of on or off signaling.

### Specificity

Specificity of coordination binding is provided by functionalyzing or decorating the carbon gate electrode. For example, many sequences of nucleic acid such as DNA or RNA will stringently coordinate or bind with the SWNT structure. These nucleic acids may be naturally occurring or synthetic. The ringed structures of the nucleic acids or other molecules such as peptides containing a large fraction of ringed structures associate strongly with the nano-tubular structures. These functionalizing or decorating additions to the SWNTs serve to selectively capture proximal molecules. When the chemical geometry is thus changed, the gating characteristic of the associated carbon bridging the input and output electrodes is modulated. A single element may be associated with a single sequence or a plurality of functionalizing sequences. Output characteristics of gating in response to one or more gaseous compounds, e.g., VOCs are then collated into a data library. When that NSE responds in the same manner, presence of the VOC is confirmed. Stringent selection of element functionalizations, and subsequent application of the controllable assay variables can optimize certainty of VOC identification at a desired level, for example, increasing manipulation of the variable parameters can achieve certainty of 99+%. In special circumstances, for example to develop rapid profiling of a new VOC signature (I.e., pathogen), a simplified screening protocol or developmental process may begin with a lower level of certainty, e.g., 85%, 95%, etc. Subsequent refinements then could be applied to raise the level of certainty until reaching a mathematical and chemical sensitivity to an acceptable level, e.g., a 99+% certainty.

A single element may be capable of indicating the presence of more than one compound. For example, similar compounds may not be distinguished in their association/coordination with the element surface and therefore may in certain circumstances produce indistinguishable signals on their own. But the single element may, for example, in conjunction with one or more other elements provide definitive results with respect to the VOCs that may interact with any one element. Alternatively, the single element when operated at a different temperature, voltage or other variable may distinguish between the different compounds binding the element under static conditions. The discussion above describing the variable inputs and input patterns and different resulting outputs relates to such differentiation capabilities.

### Fast Tracked Testing and Reading

One embodiment may include a simplified assay, perhaps featuring a chip with fewer component element or element types than a mass screening formatted chip, e.g., using only a fraction of the DNA species on the general use chip. In simplified embodiments fewer parameters may be manipulated, perhaps a static system where one or more variables, such as, voltage, temperature, etc. Have a reduced range or remain constant. When Al identifies, for example, a simplified signature for a specific set of diseases or a specific disease, such as a new virus or strain of virus, the device may be instructed to operate in a simple detection mode similar to that of a +/−strip test. Chips may thus be made specific for different preferred assays or a regular chip may be used with simplified readings. Such configurations may be used, for example, to screen for specific traits that may indicate individuals at risk or not at risk or may be used as a confirmatory test indicative of a prior result, positive or negative.

A simplified data analysis may be inherent in the chip whether at the collection site, offset from the collection site or remote. For example, a circuit can be built with specific sensors in series and/or in parallel. When the circuit produces the right gating, a positive result would be output. A side circuit on the chip possibly sharing portions of the positive negative circuitry may be included as a control. In some embodiments a completed control circuit with an incomplete or open positive circuit may produce a “negative” signal. The installed chip itself may be individualized to contain a coded instruction for the machine to operate in the designated mode, e.g., an optical patch, physically slotting, an RFID, actual machine readable code, etc., may instruct the machine to operate in the preferred program manner. Such a streamlined approach can enable extremely high throughput analysis of targeted profiles.

### Shielding

As sensitivity is heightened, machine stability becomes more important. Therefore, depending on output sensitivity targets, formats of samples, formats of delivering the samples, etc., shielding is considered a major design consideration. Electromagnetic shielding to reduce potential interference can be any suitable format, e.g., conductive material such as copper, nickel, mu-metal, amorphous metal, conductive plastics, conductive paints/inks, etc. In general, the device should be protected or shielded from any influences, that interfere with performance including, but not limited to: acoustic, temperature/thermal, electromagnetic, visible, infrared, ultraviolet, radio/micro waves, magnetic, electric, etc.

### Data Storage and Analysis

Raw data may be stored in a library linked to the sample source with any other relevant information including, but not limited to: disease diagnosed, disease status, nourishment history, time of collection, place of collection, chip cartridge(s) used, volume of sample, flow rate, temperature, volume analyzed, medical history, subject preparation steps before analysis, storage and/or chain of custody conditions, medications, gender, age, etc. Such library may be stored or transmitted in any available format and process taking safety, privacy, consent, cost, relevant laws, legal jurisdiction, storage density, transit speed, etc. Into account with a goal of interfacing groups of machines in a knowledge base where each device teaches and learns from others. Preferably, the signature library utilized by the device for disease comparison is stored locally, e.g., within the VOC analyzing device where hard-wired connection is available for optimized speed and reduced potential for interference. The cloud or dedicated remote stage sites may serve as back-ups.

Thus, preferred embodiments incorporate the stored libraries within the box or box handle. The box may serve as a sensor module and com-com; some or a large portion of activities may be subbed out to the handle or wi-fi connected device(s). When desired, portions or all of the library may be remotely stored in distal locations using communication systems known in the art and protected from cyberhacking with appropriate tools, including any available format, e.g., single encrypted, double encrypted, or block-chain coded.

Files in such library may be compiled and analyzed by knowledgeable humans, but more preferably by applying machine learning and/or artificial intelligence in any combination with human input. Such processing, analysis, and comparing multiple samples with associated information may be useful for continuous expansion of the disease repertoire and the improvement of diagnostic accuracy and quality of the output data.

Assays under the present invention can recognize a new appearance of previously known signatures or appearance of a previously unknown signature. Geographic location, possibly to a single town, lab, hospital, metropolitan area, zip code etc., will allow instant designation of identification of disease activity and proposed containment zones. In these zones, rapid testing of associated individuals and contact tracing where relevant can be initiated and accomplished before the numbers of afflicted persons become burdensome or overwhelming. An outbreak, even from a previously unknown pathogen, can therefore be identified and stopped long before reaching epidemic status and hence prevent an epidemic event expanding into a pandemic. In conjunction with the identification and partitioned follow-up of the disease making a first appearance, the disease can be characterized as to source, afflicted cells, possible treatments and/or preventions, etc.

Embodiments of the invention include one or more analytical devices comprising a detector unit capable of interfacing with an analytical unit, wherein said detector unit comprises a module designed to collect a vapor phase sample for delivery to a plurality of detection surfaces capable of interacting with the vapor phase. The vapor phase sample may be collected through a tube that terminates with a probe at the tube's end distal from the detector unit with a vapor phase sample passing through the probe and tube to a zone for vapor collection in the detector unit. The detection surface comprises a nano-sensor element (NSE) layer between its input and output with the input in contact with a base powering source and the output in communication with one or more least data collectors. The NSE layer is associated with a selection component having selection specificity for one or more compounds of interest, often a VOC of biological origin. A target compound when present (in contact with or in close proximity to a selection component) selectively alters signal to the output. The output interfaces with a component that analyzes signal from the data collector to produce a report suitable for comparison to previous reports to suggest presence of a disease or condition. A device may be shaped as a box and may incorporate an intake or exhaust fan. The box can be mounted on a handle. The box may be configured as a com-com that may comprise a graphic user interface. It may feature a touch screen. A preferable touch screen is enabled to display in portrait or landscape mode. A box of the invention may be configured to mount in or may be mounted in a support cradle, e.g., a cell phone or com-com support cradle. A box and/or cradle of the invention may feature hardwire ports that connect with a com-com. Embodiments may incorporate one or more thermometer and/or hygrometer. Such thermometer and/or hygrometer participate in providing information that is used to modulate testing conditions including but not limited to charge on at least one NSE, temperature of at least one NSE, temperature of the vapor, flow rate of the vapor, etc. Various embodiments may feature selective binding, or selectively permeable, membranes for concentrating, isolating, filtering, sequestering, etc., the vapor phase sample. Devices may remain at ambient pressure and/or may feature zones whose pressure is relatively negative in comparison to an ambient pressure. Preferred NSEs comprise carbon. Preferred carbons include a graphene configuration, that may be essentially planar, irregular, patterned or curved. A preferred curved graphene structure is in the form of a single walled nanotubular carbon (SWNT). Other configurations include irregular (e.g., like sputtered), corrugated, crumpled, etc. In some embodiments, a heating element may be preferred. Such heating element may work in concert with a thermostatic and/or hygrometric device to provide a stand pressure and/or water content for the sample, chip, handle, etc. Temperature and humidity are thereby variably controlled. Temperature control may comprise the whole device or box; it may be directed at a chip or chip; Local control of individual or blocks (plurality of) NSEs may be featured. Devices may feature physical components that may shield the sensing chamber, the user, the vapor source, etc., from undesired physical or publicity (see e.g., privacy) interactions. Some embodiments may incorporate a prep chamber or device to direct the vapor phase at a standardized pressure before crossing said detection surface. The prep or control chamber may include heating or cooling for delivering standard vapor phase. Vapor flow may be diffuse and/or connective. Convective flow may involve a pump or temperature differential.

1. An analytical device comprising a detector unit capable of interfacing with an analytical unit, said detector unit comprising a module designed to collect a vapor phase sample for delivery to a plurality of detection surfaces capable of interacting with said vapor phase; said vapor phase sample being collected through a tube; said tube terminating with a probe at said tube's distal end from said detector unit said vapor phase sample passing through said probe and tube for collection in said detector unit;
said detection surface comprising a nano-sensor element (NSE) layer between an input and an output; said input in contact with a base power source;
said output in communication with at least one data collector; said NSE layer associated with a selection component with selection specificity for one or more compounds of interest; said selection component when in contact with or in close proximity to a target compound possibly present in said vapor phase that when present selectively alters signal to said output; and a component for analyzing said signal from said data collector and producing a report suitable for comparison to previous reports.

2. The device of claim 1 wherein said selection specificity comprises at least one of an attractive component and a repulsive component.

3. The device of claim 1 having a surface area designed to interface with a site selected from the group consisting of: axilla, otic area, body orifice, scalp, and selected zone of skin.

4. The device of claim 3 wherein said surface area comprises the tip of a probe.

5. The device of claim 1 wherein a said vapor phase sample is delivered to said detection surface in a plurality of pulses.

6. The device of claim 1 further comprising a physical component that shields at least one of the device, environment, operator, and/or vapor source, from at least one other.

7. The device of claim 6 wherein said shielding component is selected from the group consisting of: acoustic shielding, temperature/thermal shielding, electromagnetic shielding, visible light shielding, infrared light shielding, ultraviolet light shielding, radio/micro wave shielding, magnetic shielding, electric shielding, vibration shielding and unauthorized use shielding.

8. The device of claim 7 comprising electromagnetic shielding that comprises conductive material.

9. The device of claim 8 wherein said conductive material is selected from the group consisting of: copper, nickel, mu-metal, amorphous metal, conductive plastics, conductive paints and conductive inks.

10. The device of claim 1 wherein at least one molecule in the vapor phase has its position under control of at least one force selected from the group consisting of: photo momentum, acoustic, convective gas flow, magnetic, electromagnet, electric, and Lorentz force.

11. The device of claim 1 wherein said NSE has a shape selected from the group consisting of: rectangular, rhomboid, hexagonal, triangular, elliptical, circular, irregular, crumpled and creviced.

12. The device of claim 11 wherein said NSE comprises a graphene layer.

13. The device of claim 12 wherein said graphene layer comprises a curved graphene.

14. The device of claim 13 Wherein said curved graphene is formed as a single walled nanotubular carbon configuration.

15. The device of claim 1 further
comprising a detector unit capable of interfacing with an analytical unit, said detector unit comprising a plurality of detection surfaces capable of interacting with an ambient vapor phase; said detection surface comprising a Field Effect Transistor nano-sensor element (NSE) layer between an input and an output electrode; said input electrode in contact with a base or feeder voltage source; said output electrode in contact with at least one data collector; said FET layer associated with a selection component with selection specificity for one or more compounds of interest; said selection component when in contact with a target compound possibly present in said vapor phase selectively gating current across said detection surface thereby altering signal to said output electrode; and a component for collecting said signal to said output electrode.

16. A method for determining disease status of a selected subject, said method comprising: accessing a device according to claim 1; guiding the probe end to a selected area or zone of said subject; switching said device to collect a vapor phase sample; allowing said device to assay and analyze said vapor phase sample for comparison with one or more disease signature libraries.

17. The method of claim 16
wherein said probe is one selected from the group consisting of: an otoscope, a cystoscope, a colposcope, a vaginal endoscope, an esophageal endoscope, a colonoscope, a nasal endoscope, and a multi-purpose scope.

18. The method of claim 16 wherein said vapor phase sample is collected in a medical setting.

19. The method of claim 16 wherein said vapor phase sample is collected at an entrance to a home or business.

20. A method for determining a
subject's disease status relating to a disease of interest, said method comprising: a) placing an analytical device in accordance with claim 1 within the or immediately outside an otic canal
of said subject; b) collecting a sample from said otic canal
within said analytical device; c) analyzing said sample using said device to output data; d) collecting and analyzing the data outputted from sensors within said device to produce a subject signature; e) comparing said subject signature to a dataset comprising at least one signature associated with at least one disease of interest; and f) reporting a match or lack or match between said subject signature and said at least one signature associated with at least one disease of interest.
